SEQUENCE LISTING (11 sequences)

SEQ ID NO: 1 - DNA, 2339 bp, Homo sapiens
cctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccaaaaaaaaaaa

SEQ ID NO: 2 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 3 - DNA, 1740 bp, Homo sapiens
ccagcgttagcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaaaaaaaaa

SEQ ID NO: 4 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

SEQ ID NO: 5 - DNA, 21 bp, Artificial Sequence
  synthetic
gcctggcctgaatattatact

SEQ ID NO: 6 - DNA, 21 bp, Artificial Sequence
  synthetic
ggaatccggaataaatactac

SEQ ID NO: 7 - DNA, 21 bp, Artificial Sequence
  synthetic
gcctggcctgaatattatact

SEQ ID NO: 8 - DNA, 21 bp, Artificial Sequence
  synthetic
ggataatggtgattgagatgg

SEQ ID NO: 9 - DNA, 21 bp, Artificial Sequence
  synthetic
gtggacatccagctaaagtat

SEQ ID NO: 10 - DNA, 21 bp, Artificial Sequence
  synthetic
atctttgacaagcactataag

SEQ ID NO: 11 - DNA, 21 bp, Artificial Sequence
  synthetic
ggataatggtgattgagatgg

(19) United States
(12) Patent Application Publication
(10) Pub. No.: US 2016/0312311 A1
(43) Pub. Date: Oct. 27, 2016

(54) MARKERS FOR ISOCITRATE DEHYDROGENASE INHIBITORS

(71) Applicants: Fallon LIN, (US); Young Shin CHO, Cambridge, MA (US); Julian Roy LEVELL, Cambridge, MA (US); Margaret Elise MCLAUGHLIN, Cambridge, MA (US); Ronald MEYER, Cambridge, MA (US); Raymond PAGLIARINI, Cambridge, MA (US); Veronica SAENZ-VASH, Cambridge, MA (US); Olga SHEBANOVA, Cambridge, MA (US); Huili ZHAI, Cambridge, MA (US)

(72) Inventors: Young Shin Cho, Cambridge, MA (US); Julian Roy Levell, Arlington, MA (US); Fallon Lin, Boston, MA (US); Margaret Elise McLaughlin, Cambridge, MA (US); Ronald Meyer, East Sandwich, MA (US); Raymond Pagliarini, Arlington, MA (US); Veronica Saenz-Vash, Cambridge, MA (US); Olga Shebanova, Somerville, MA (US); Huili Zhai, Cambridge, MA (US)

(73) Assignee: NOVARTIS AG, Basel (CH)

(21) Appl. No.: 14/772,976

(22) PCT Filed: Feb. 24, 2014
(86) PCT No.: PCT/US2014/018075

§ 371 Date: Sep. 4, 2015

Related U.S. Application Data
(60) Provisional application No. 61/777,121, filed on Mar. 12, 2013.

(51) Int. Cl. C12Q 1/68; G01N 33/68

(52) U.S. Cl. CPC C12Q 1/6888; G01N 33/6875; G01N 33/6848; C12Q 2600/156; G01N 2333/47; G01N 2800/7028; G01N 2800/52; G01N 2333/904; C12Q 2600/158; C12Q 2600/154; C12Q 2600/106

(57) ABSTRACT

The invention provides methods of detecting cancer and detecting activity of IDH inhibitors, and methods of screening for IDH inhibitors by detecting levels of H3K9me2.

### FIELD OF THE INVENTION

The present disclosure relates to the field of pharmacogenomics, and the use of biomarkers useful in detecting cancer cells in a patient, detecting patient response to Isocitrate Dehydrogenase inhibitors and screening of compounds.

### BACKGROUND

Isocitrate dehydrogenase (IDH) is a key family of enzymes found in cellular metabolism. They are NADP⁺/NAD⁺ and metal dependent oxidoreductases of the enzyme class EC 1.1.1.42. The wild type proteins catalyze the oxidative decarboxylation of isocitrate to alpha-ketoglutarate generating carbon dioxide and NADPH/NADH in the process. They are also known to convert oxalosuccinate into alpha-ketoglutarate. Mutations in IDH1 (cytosolic) and IDH2 (mitochondrial) have been identified in multiple cancer types including, but not limited to, glioma, glioblastoma multiforme, paraganglioma, supratentorial primordial neuroectodermal tumors, acute myeloid leukemia (AML), prostate cancer, thyroid cancer, colon cancer, chondrosarcoma, cholangiocarcinoma, peripheral T-cell lymphoma, and melanoma. (See L. Deng et al., Trends Mol. Med., 2010, (16): 387; T. Shibata et al., Am. J. Pathol., 2011, 178(3):1395; Gaal et al., J. Clin. Endocrinol. Metab. 2010 95(3):1274; Hayden et al., Cell Cycle, 2009 (8):1806; Balss et al., Acta Neuropathol., 2008 (116):597). The mutations have been found at or near key residues in the active site: G97D, R100, R132, H133Q, and A134D for IDH1, and R140 and R172 for IDH2. (See L. Deng et al., Nature, 2009, (462): 739; L. Sellner et al., Eur. J. Haematol., 2011 (85): 457).

These mutant forms of IDH are shown to have a neomorphic activity (also known as a gain of function activity), reducing alpha-ketoglutarate to 2-hydroxyglutarate (2-HG). (See P. S. Ward et al., Cancer Cell, 2010, (17):225) In general, production of 2-HG is enantiospecific, resulting in generation of the D-enantiomer (also known as R enantiomer or R-2-HG). Normal cells have low native levels of 2-HG, whereas cells harboring these mutations in IDH1 or IDH2 show significantly elevated levels of 2-HG. High levels of 2-HG have been detected in tumors harboring the mutations. For example, high levels of 2-HG have been detected in the plasma of patients with mutant IDH containing AML. (See S. Gross et al., J. Exp. Med., 2010, 207(2): 339). High levels of 2-HG are highly associated with tumorigenesis.

Mutant IDH2 is also associated with the rare neurometabolic disorder D-2-hydroxyglutaric aciduria type II (D-2-HGA type II). Germline mutations were found at R140 in IDH2 in 15 patients having D-2-HGA type II. Patients having this disorder also have consistently increased levels of D-2-HG in their urine, plasma and cerebrospinal fluid. (See Kranendijk, M. et al., Science, 2010 (330): 336). Finally, patients with Ollier Disease and Mafucci Syndrome (two rare disorders that predispose to cartilaginous tumors) have been shown to be somatically mosaic for IDH1 and 2 mutations and exhibit high levels of D-2-HG. (See Amary et al., Nature Genetics 2011 43(12):1262 and Pansuriya et al., Nature Genetics 2011 43(12):1256).

Mutations in IDH that are neomorphic and generate 2-HG also generate epigenetic modifications, specifically histone methylation modifications. By using these epigenetic changes as a biomarker ensures that the correct patients receive the appropriate treatment and during the course of the treatment the patient response to IDH inhibitors can be determined. Measuring the response of cancers to IDH inhibitors by utilizing biomarkers such as change in the levels of histone methylation will aid in understanding the mechanism of action which has not been addressed using IDH inhibitors (Hull-Ryde et al., Keystone Symposium abstract #X42009, Feb. 24-Mar. 1, 2013). This allows for a more timely and aggressive treatment as opposed to a trial and error approach.

### SUMMARY OF THE INVENTION

The disclosure is directed to diagnosis of cancer by analysis of histone methylation changes by IDH mutations. IDH mutational analysis and/or histone methylation analysis provides a “signature” for cancer that has increased accuracy and specificity in segregating cancer patients. The method analyzes the change in level of di-methylation of histone H3 at lysine 9 (H3K9me2) in a cancer sample taken from a patient and then compared to a non-mutant or wild-type control. The level of H3K9me2 change can be indicative of a favorable response or an unfavorable one. The invention is an example of “personalized medicine” wherein patients are treated based on a functional genomic signature that is specific to that individual.

The predictive value of change in the level of H3K9me2 can also be used after treatment with an IDH inhibitor to determine if the patient is responsive to the treatment. Once an IDH inhibitor has been administered, the changes in level of H3K9me2 can be assayed to monitor the continued response of the patient to the therapy. This is useful in determining that patients receive the correct course of treatment. The disclosure provides a method of assaying for a patient response to an IDH inhibitor.

### DESCRIPTION OF THE DRAWINGS

FIG. 1A/B shows that IDH1, IDH2 mutations and an increase in 2-HG levels increase levels of H3K9me2.

FIG. 2A/B shows a Western blot of IDH1 mutant knockdown with shRNA or with a specific IDH1 inhibitor, and a decrease in level of H3K9me2 with both types of inhibitor.

FIG. 3 is a Western blot demonstrating that H3K9me2 methylation level is reduced by knockdown with two IDH2 specific shRNA (shIDH2-309 and shIDH2-891)

FIG. 4 demonstrates that a specific IDH1 inhibitor does not affect H3K9me2 in an IDH1 WT genotype.

FIG. 5 shows that increasing concentrations of an IDH1 inhibitor reduces levels of H3K9me2 in an IDH2 WT background, but not in an IDH2 neomorphic mutant background.

FIG. 6A/B shows that the reduction of H3K9me2 levels by an IDH1 inhibitor is detectable by immunohistochemistry.

FIG. 7 is a heat map depicting a histone profile of several cell lines upon treatment with IDH1 inhibitor or IDH2 knockdown across several different histones and histone methylation sites.

### DESCRIPTION OF THE INVENTION

The disclosure provides for a method of detecting cancer in a patient, the method comprising: a) obtaining a cancer sample from a patient; b) sequencing for the presence of a isocitrate dehydrogenase (IDH) mutation in the cancer sample; c) comparing the IDH mutation sequence to an IDH sequence in a non-cancerous or normal patient sample; and d) assaying for the level of di-methylation (me2) of histone H3 at lysine 9 (H3K9me2) in the cancer sample with an IDH mutation and comparing it with the level of H3K9me2 of a non-cancerous or normal patient sample, and a higher level of H3K9me2 in the cancer sample compared to the non-cancerous or normal patient sample is indicative of cancer.

The method wherein the IDH mutation is a mutation in IDH1 and is an arginine to histidine change at amino acid position 132 (IDH1-R132H).

The method wherein the IDH mutation is a mutation in IDH1 and is an arginine to cysteine change at amino acid position 132 (IDH1-R132C).

The method wherein the IDH mutation is a mutation in IDH2 and is an arginine to lysine change at amino acid position 172 (IDH2-R172K).

The method wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

A method of assaying for the response of a patient to treatment with an IDH inhibitor, the method comprising: a) obtaining a cancer sample from a patient prior to administration of an IDH inhibitor; b) administration to a patient of at least one IDH inhibitor; c) assaying for a level of H3K9me2 in the sample obtained from the patient who has been administered the IDH inhibitor; and d) comparing the level of H3K9me2 in the cancer sample taken prior to administration of the IDH inhibitor or the level of H3K9me2 in a non-cancerous or control sample.

The method wherein the level of H3K9me2 is reduced.

The method wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

The method wherein the IDH inhibitor inhibits IDH1.

The method wherein the IDH inhibitor inhibits an IDH1 mutant, and the IDH1 mutation is an arginine to histidine change at amino acid position 132 (IDH1-R132H).

The method wherein the IDH inhibitor inhibits an IDH1 mutant, and the IDH1 mutation is an arginine to cysteine change at amino acid position 132 (IDH1-R132C).

The method wherein the IDH inhibitor is an oxazolidinone.

The method wherein the IDH inhibitor inhibits an IDH2 mutant, and the IDH2 mutation is an arginine to lysine change (IDH2-R172K).

The method wherein the IDH inhibitor is administered at different time points.

The method wherein assaying for the level of H3K9me2 in the cancer sample is measured at least at two different time points.

The method wherein the steps c) and d) are repeated at 1 hour, 2 hours, 3, hours, 4, hours, 8 hours, 16 hours and 48 hours.

The method wherein assaying for the level of H3K9me2 is done by mass spectrometry.

The method wherein assaying for the level of H3K9me2 is done by Western blotting.

A method of screening for an IDH inhibitor candidate, the method comprising: a) contacting a cell containing an IDH mutation with an IDH inhibitor candidate; b) assaying for a level of H3K9me2; and c) comparing the level of H3K9me2 from the IDH mutant cell contacted with the IDH inhibitor candidate with the level of H3K9me2 of a normal or control cell and/or untreated cell containing the IDH mutation.

The method wherein the IDH inhibitor inhibits IDH1.

The method wherein the IDH inhibitor inhibits an IDH1 mutant, and the IDH1 mutation is an arginine to histidine change at amino acid position 132 (IDH1-R132H).

The method wherein the IDH inhibitor inhibits an IDH1 mutant, and the IDH1 mutation is an arginine to cysteine change at amino acid position 132 (IDH1-R132C).

The method wherein the IDH inhibitor inhibits an IDH2 mutant, and the IDH2 mutation is an arginine to lysine change (IDH2-R172K).

The method wherein the cell containing an IDH mutation is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

The method wherein assaying for the level of H3K9me2 is done by mass spectrometry.

The method wherein assaying for the level of H3K9me2 is done by Western blotting.

A composition comprising H3K9me2 for use in diagnosing a patient response in a selected cancer patient population, wherein the cancer patient population is selected on the basis of (i) having increased levels of H3K9me2 in a cancer cell sample obtained from said patients compared to a normal control cell sample, and (ii) H3K9me2 levels are is reduced upon administration of an IDH inhibitor.

The composition wherein the IDH inhibitor inhibits IDH1.

The composition wherein the IDH inhibitor inhibits an IDH1 mutant, wherein the mutation in IDH1 is an arginine to histidine change at amino acid position 132 (IDH1-R132H).

The composition wherein the IDH inhibitor inhibits an IDH1 mutant, wherein the mutation in IDH1 is an arginine to cysteine change at amino acid position 132 (IDH1-R132C).

The composition wherein the IDH inhibitor inhibits an IDH2 mutant, wherein the mutation in IDH2 is an arginine to lysine change (IDH2-R172K).

The composition wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

A kit for predicting the response of a cancer patient to treatment with an IDH inhibitor comprising: i) means for detecting H3K9me2; and ii) instructions how to use said kit.

Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

The terms “marker” or “biomarker” are used interchangeably herein. A biomarker can be without limitation: nucleic acid or polypeptide expression; an epigenetic change such as histone methylation or protein phosphorylation and the presence or absence of the biomarker used to detect a specific cancer type or detect a response to a therapeutic. For example, di-methylation of histone H3 at the lysine at position 9 (H3K9me2) is a biomarker in a cancer cell containing an IDH mutation, when its level is increased as compared to H3K9me2 in normal (non-cancerous) cell or control cell. In another example, H3K9me2 is a biomarker when its levels are reduced upon administration of an IDH inhibitor to a cancer cell containing an IDH mutation.

A cell is “responsive” or displays “responsiveness” to inhibition with an IDH inhibitor when the H3K9me2 level is reduced compared to wild type H3K9me2 level.

“IDH” refers to an isocitrate dehydrogenase gene. Unless specifically stated otherwise, IDH as used herein, refers to human IDH. There are two isoforms of IDH, IDH1 and IDH2. IDH1 has been assigned accession number NM_005896.2 (DNA (SEQ ID NO. 1)) and (protein (SEQ ID NO.2)). IDH2 has been assigned accession number NM_002168.2 (SEQ ID NO. 3) and (protein (SEQ ID NO.4)).

A “mutant” or “mutation” is any change in DNA or protein sequence that deviates from wild type IDH. This includes single base DNA changes, single amino acid changes, multiple base changes in DNA and multiple amino acid changes. This also includes insertions, deletions and truncations of an IDH gene and its corresponding protein. For example, an IDH1 mutation can be an argenine to cysteine change at amino acid position 132 (IDH1-R132C).

“Methylation” is the modification of amino acids on a histone protein by the addition of a methyl group. The amino acid can have no methylation (me0), have a single methyl group added (me1), two methyl groups added (me2) or three methyl groups (me3). For example, the nomenclature “H3K9me2” indicates that 2 methyl groups were added to histone H3 to the lysine at position 9. The “methylation status” or “methylation profile” refers to the histone, the amino acid and 0-3 methyl group modifications (me0-me3).

“Differential methylation” refers to the change in level of a methylated histone form. For example, differential methylation can be when there is 40% H3K9me2 in an untreated cancer cell containing an IDH mutation, and upon treatment with an IDH inhibitor the level of H3K9me2 in the cell is reduced to 25%.

As used herein the term “neomorphic activity” refers to a gain of function or novel activity of a protein that the wild-type protein does not have or does not exhibit to a significant degree. For example, a neomorphic activity associated with a mutant form of IDH1 and IDH2 is the ability to reduce alpha-ketoglutarate to 2-hydroxyglutarate (i.e. 2-HG, specifically R-2-HG). The wild type form of IDH1 and IDH2 does not have the ability to reduce alpha-ketoglutarate to 2-hydroxyglutarate (i.e. 2-HG, specifically R-2-HG) or if it does have this ability, it does not produce significant (i.e. harmful or disease causing) amounts of 2-HG. “Inhibitors” of IDH neomorphic activity can be without limitation: shRNA, RNAi, members of the oxazolidinone class of compounds, for example, compound 162, or any molecule which has the ability to inhibit the neomorphic production of 2-HG by IDH1 or IDH2 mutants. An “inhibitor” of IDH as used herein reduces the level of H3K9me2 in the cell.

A “control cell,” “normal cell” or “wild-type” refers to a non-cancerous cell.

A “control tissue,” “normal tissue” or “wild-type” refers to a non-cancerous tissue.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and may perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labelling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

“Sequencing” refers to obtaining sequence information from a nucleic acid strand, generally by determining the identity of nucleotides within a specific nucleic acid molecule. While in some instances, sequencing a given region of a nucleic acid molecule includes identifying each and every nucleotide within the region that is sequenced, in some instances, only particular nucleotides of interest in the region are determined, while the identity of some nucleotides remains undetermined. Any suitable method of sequencing may be used, for example, labeled or dye-containing nucleotide or fluorescent based nucleotide sequencing methods.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

“Gene expression” or alternatively a “gene product” refers to the nucleic acids or amino acids (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the histone, polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. As is apparent to those of skill in the art, a non-naturally occurring histone, polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in PCR: A Practical Approach, M. MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (Sambrook et al., Molecular Cloning: A Laboratory Manual, 2^{nd} edition (1989)).

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non-cancerous cell or tissue) is undetectable.

A high expression level of the gene may occur because of over expression of the gene or an increase in gene copy number. The gene may also be translated into increased protein levels because of deregulation or absence of a negative regulator.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories), polyHIPEI™ resin (obtained from Aminotech, Canada), polyamide resin (obtained from Peninsula Laboratories), polystyrene resin grafted with polyethylene glycol (TentaGelR™, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087 and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity can be assessed by measuring levels of messenger RNA using a gene chip such as the Affymetrix® HG-U133-Plus-2 GeneChips (Affmetrix, Santa Clara, Calif.). High-throughput, real-time quantitation of RNA of a large number of genes of interest thus becomes possible in a reproducible system.

The terms “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68° C. or 0.015M sodium chloride, 0.0015M sodium citrate, and 50% formamide at 42° C. (see Sambrook, supra). An example of a “moderately stringent” hybridization is the conditions of: 0.015M sodium chloride, 0.0015M sodium citrate at 50-65° C. or 0.015M sodium chloride, 0.0015M sodium citrate, and 20% formamide at 37-50° C. The moderately stringent conditions are used when a moderate amount of nucleic acid mismatch is desired. One of skill in the art will appreciate that washing is part of the hybridization conditions. For example, washing conditions can include 02.×-0.1×SSC/0.1% SDS and temperatures from 42-68° C., wherein increasing temperature increases the stringency of the wash conditions.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures. Cancer can include without limitation: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

“Suppressing” tumor growth indicates a reduction in tumor cell growth when contacted with a chemotherapeutic compared to tumor growth without a chemotherapeutic agent. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage.

A “composition” is a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives, for example; proteins, peptides, amino acids, lipids, and carbohydrates (e.g., sugars, including monosaccharides and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carbohydrate excipients include, for example; monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “carrier” further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-quadrature-cyclodextrin), polyethylene glycols, antimicrobial agents, sweeteners, antioxidants, antistatic agents, surfactants (e.g., polysorbates such as TWEEN 20™ and TWEEN 80™), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Remington's Pharmaceutical Science., 15^{th} Ed. (Mack Publ. Co., Easton (1975) and in the Physician's Desk Reference, 52^{nd} ed., Medical Economics, Montvale, N.J. (1998).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

Detection of IDH Mutations

The detection of IDH mutations can be done by any number of ways, for example: DNA sequencing, PCR based methods, including RT-PCR, microarray analysis, Southern blotting, Northern blotting and dip stick analysis.

The polymerase chain reaction (PCR) can be used to amplify and identify IDH mutations from either genomic DNA or RNA extracted from tumor tissue. PCR is well known in the art and is described in detail in Saiki et al., Science 1988 (239):487 and in U.S. Pat. No. 4,683,195 and U.S. Pat. No. 4,683,203.

Methods of detecting IDH mutations by hybridization are provided. The method comprises identifying an IDH mutation in a sample by contacting nucleic acid from the sample with a nucleic acid probe that is capable of hybridizing to nucleic acid with an IDH mutation or fragment thereof and detecting the hybridization. The nucleic acid probe is detectably labeled with a label such as a radioisotope, a fluorescent agent or a chromogenic agent. Radioisotopes can include without limitation; ³H, ³²P, ³³P and ³⁵S etc. Fluorescent agents can include without limitation: fluorescein, texas red, rhodamine, etc.

The probe used in detection that is capable of hybridizing to nucleic acid with a IDH mutation can be from about 8 nucleotides to about 100 nucleotides, from about 10 nucleotides to about 75 nucleotides, from about 15 nucleotides to about 50 nucleotides, or about 20 to about 30 nucleotides. The probe or probes can be provided in a kit, which comprise at least one oligonucleotide probe that hybridizes to or hybridizes adjacent to an IDH mutation. The kit can also provide instructions for analysis of patient cancer samples that can contain a IDH mutation.

Single stranded conformational polymorphism (SSCP) can also be used to detect IDH mutations. This technique is well described in Orita et al., PNAS 1989, 86:2766-2770.

Antibodies directed against IDH can be useful in the detection of cancer and the detection of mutated forms of IDH. Antibodies can be generated which recognize and specifically bind only a specific mutant form of IDH and do not bind (or weakly bind) to wild type IDH. These antibodies would be useful in determining which specific mutation was present and also in quantifying the level of IDH protein. For example, an antibody can be directed against the arginine to histidine change at amino acid position 132 (R132H) of IDH1. An antibody that recognizes this amino acid change and does not specifically bind to wild type IDH1 could identify the specific mutation by Western blotting. Such antibodies can be generated by using peptides containing a specific IDH mutation.

Measurement of Gene Expression

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix∩ U133 microarray chips (Affymax, Santa Clara, Calif.).

In one aspect, gene expression is detected and quantitated by hybridization to a probe that specifically hybridizes to the appropriate probe for that gene. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™ Life Technologies, Carlsbad, Calif.), fluorescent dyes (e.g., fluorescein, texas red, rhodamine, green fluorescent protein, and the like), radiolabels (e.g., 3^{H}, 125^{I}, 35^{S}, 14^{C}, or 32^{P}) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see: Laboratory Techniques in Biochemistry and Molecular Biology, Vol. 24: Hybridization with Nucleic Acid Probes, P. Tijssen, ed. Elsevier, N.Y. (1993).

Detection of Polypeptides

An IDH mutation when translated into protein can be detected by specific antibodies. A mutation in an IDH protein can change the antigenicity, so that an antibody raised against a IDH mutant antigen (e.g. a specific peptide containing a mutation) will specifically bind the mutant IDH and not recognize the wild-type.

Expression level of an IDH mutant can also be determined by examining protein expression. Determining the protein level involves measuring the amount of any immunospecific binding that occurs between an antibody that selectively recognizes and binds to an IDH mutant in a sample obtained from a patient and comparing this to the amount of immunospecific binding of an IDH protein in a control sample. The amount of protein expression of an IDH mutant protein can be increased or reduced when compared with control expression. A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, confocal microscopy, enzymatic assays, surface plasmon resonance and PAGE-SDS.

Detection of Histone Methylation Changes

Histones can be extracted from the selected cell lines or engineered derivative cell lines as described (Thomas et al., J. Prot. Res. 2006 (5): 240-247). In parallel, histones can be extracted from cell lines cultivated using SILAC (Ong et al., Mol. Cell. Prot. 2002 (1): 376-386). A standardization mixture consisting of equal parts of the SILAC histones from cell lines can be formulated. In turn, equal parts of the standardization mixture and histones from each selected cell line or engineered derivative cell line can be formulated to constitute each “sample.” Histone peptides can be prepared from each sample as described (Garcia et al., Nat. Protoc. 2007 2, 933-938). Peptide samples can be analyzed using liquid chromatography-mass spectrometry (LCMS) with a Q-Exactive hybrid quadrupole-electrostatic trap mass spectrometer using an acquisition method designed to isolate each peptide species of interest and cause it to be dissociated by collisional activation into fragment ions. Each peptide species of interest can be quantified using Skyline® software (MacLean et al., Bioinfo. 2010 (26):966-968) wherein the LCMS peak area ratios of the intensities of the fragment ions of each peptide in the selected cell line and the standardization mix can be determined. These ratios can be further normalized in each sample to the total amount of histones present in the cell line-derived sample and the standardization mix as determined by LCMS.

Histone methylation can also be detected by Western blotting, as anti-histone antibodies are commercially available (Abcam, Cambridge, Mass.). Western blot analysis and related methods can also be used to detect and quantify the presence of methylated histones in a sample. The Western technique generally involves separating sample products by gel electrophoresis on the basis of molecular weight and transferring the separated products to a suitable membrane, (for example: a nitrocellulose filter, a nylon filter, or PVDF filter) (see Harlow and Lane (1988) Antibodies, A Laboratory Manual, Cold Spring Harbor Publications, New York). The membrane is then incubated in a solution containing a primary antibody that will bind to the methylated histones. There is a washing step to remove non-specifically bound antibodies, and the membrane is incubated again with a secondary antibody which will specifically bind to the primary antibody. The secondary antibodies are directly labeled or alternatively are subsequently detected using other labeled antibodies. The secondary antibody can also be detected if the secondary antibody is radiolabled or enzymatically labeled and can be reacted with a substrate. The Western blot can then be quantified.

Assaying for Biomarkers

Once a patient has been determined to have an IDH mutation, administration of a IDH inhibitor to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

Compound 162 is a member of the oxazolidinone (3-pyrimidinyl-4-yl-oxazolidin-2-one) family, and is a specific inhibitor of the neomorphic activity of IDH1 mutants and its structure is show below and has the chemical name (S)-4-isopropyl-3-(2-(((S)-1-(4 phenoxyphenyl)ethyl)amino)pyrimidin-4-yl)oxazolidin-2-one. Compound 162 and family members are the subject of U.S. 61/539,553 (see Example 162). U.S. 61/539,553 discloses in Table 30 that 604 compounds of the oxazolidinone family were tested for their ability to inhibit an IDH1 neomorphic mutant (IDH1-R132H) in a LC-MS biochemical assay and in a fluorescence biochemical assay.

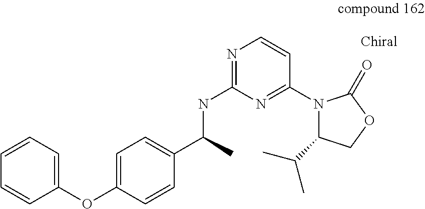 [SMILES: CC(C)[C@H]1COC(=O)N1c1ccnc(N[C@@H](C)c2ccc(Oc3ccccc3)cc2)n1]

Levels H3K9me2 can be assayed for after administration of an IDH inhibitor in order to determine the response of the cancer cell. In addition, H3K9me2 levels can be assayed for in multiple timepoints after a single IDH inhibitor administration. For example, after an initial bolus of a IDH inhibitor is administered, H3K9me2 levels can be assayed for at 1 hour, 2 hours, 3 hours, 4 hours, 8 hours, 16 hours, 24 hours, 48 hours, 3 days, 1 week, 1 month or several months after the first administration.

H3K9me2 levels can be assayed for after each IDH inhibitor administration, so if there are multiple IDH inhibitor administrations, then assaying for H3K9me2 levels after each administration can determine the continued course of treatment. The patient could undergo multiple IDH inhibitor administrations and then H3K9me2 levels can be examined at different timepoints. For example, a course of treatment may require administration of an initial dose of IDH inhibitor, a second dose a specified time period later, and still a third dose. H3K9me2 could be assayed for at 1 hour, 2 hours, 3 hours, 4 hours, 8 hours, 16 hours, 24 hours, 48 hours, 3 days, 1 week, 1 month or several months after administration of each dose of H3K9me2.

Finally, there is administration of different IDH inhibitors, for example an IDH1 inhibitor could be followed by an IDH2 inhibitor, if both mutations are present, followed by assaying for H3K9me2 levels. In this embodiment, more than one IDH inhibitor is chosen and administered to the patient. H3K9me2 levels can then be assayed for after administration of each different IDH inhibitor. This assay can also be done at multiple timepoints after administration of the different IDH inhibitor. For example, a first IDH inhibitor could be administered to the patient and H3K9me2 levels assayed for at 1 hour, 2 hours, 3 hours, 4 hours, 8 hours, 16 hours, 24 hours, 48 hours, 3 days, 1 week, 1 month or several months after administration. A second IDH inhibitor could then be administered and H3K9me2 levels can be assayed for again at 1 hour, 2 hours, 3 hours, 4 hours, 8 hours, 16 hours, 24 hours, 48 hours, 3 days, 1 week, 1 month or several months after administration of the second IDH inhibitor.

Kits for assessing H3K9me2 levels can be made. For example, a kit comprising antibodies can be used for assessing H3K9me2 levels either by immunohistochemistry or by Western blot. Alternatively, a kit containing reagents to analyze H3K9me2 by mass spectrometry would be a useful alternative to antibody based methods.

Screening for IDH Inhibitors

It is possible to use IDH mutations and analysis of H3K9me2 to screen for additional IDH inhibitors. This method comprises choosing or engineering a cell with an IDH mutation (e.g. IDH1-R132H), the cell is then contacted with the candidate IDH inhibitor compound and the contacted cell is assayed. As the IDH mutation is neomorphic, it results in increased H3K9me2 levels and assaying for a reduction in H3K9me2 when compared to H3K9me2 in a control cell would indicate that the candidate compound is an IDH inhibitor. In addition to the reduction in H3K9me2 levels, the contacted cell can also be assayed for reduction in 2-HG levels or an increase in apoptosis.

### EXAMPLES

### Example 1

### IDH1, IDH2 Mutations and 2-HG Addition Increase Methylation of H3K9

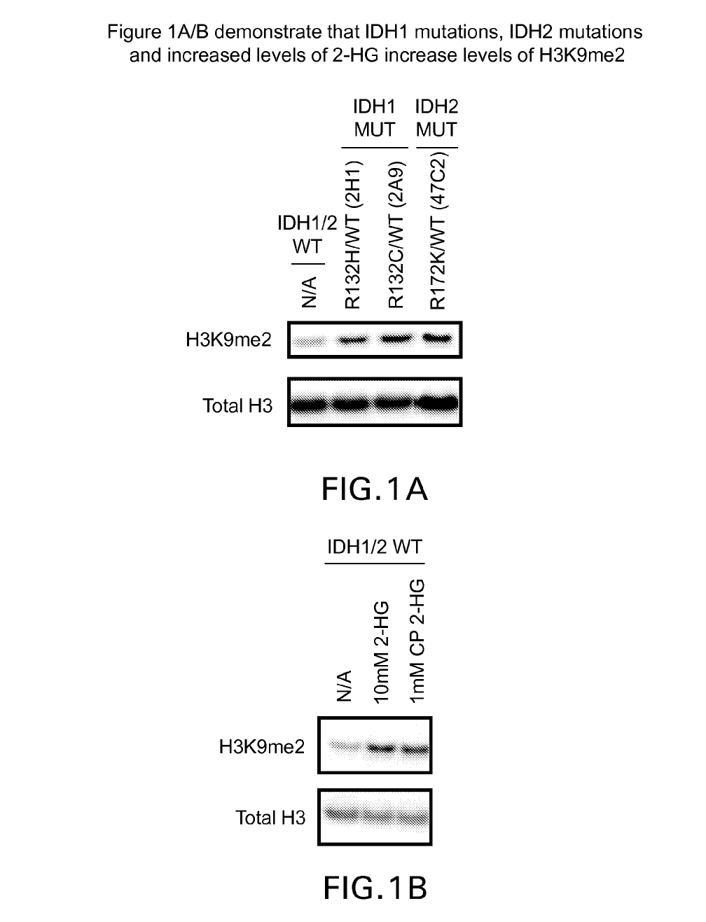

In order to evaluate the effect of IDH1 mutations, IDH2 mutations and 2-HG on H3K9me2, HCT116 colorectal cells that were engineered to endogenously express various clinically relevant mutations in IDH1 or IDH2 (Horizon Discovery, Cambridge, UK) were assayed for changes in H3K9me2 levels. As shown in FIG. 1, the isogenic cell lines tested are as follows, with relevant clone identities in parentheses: HCT116 IDH1 and IDH2 WT (“parental”), HCT116 IDH1-R132H/WT (2H1), HCT116 IDH1-R132C/WT (2A9), and HCT116 IDH2-R172K/WT (47C2). HCT116 IDH1/2 WT cells were also exogenously treated with 10 mM 2-HG or 1 mM Cell-Permeable 2-HG for about 30 days. All cells were grown in McCoy's 5A Media (Life Technologies, Carlsbad, Calif.) containing 10% Fetal Bovine Serum (Hyclone, Logan, Utah). At least 10⁶ cells per line were harvested and subject to a Histone Acid Extraction® Protocol (Abcam, Cambridge, Mass.). Cells in culture were rinsed with PBS and detached using 0.25% trypsin. Trypsin was deactivated using fresh media and the sample was spun down at 1000 rpm for 5 minutes. Media was aspirated and samples were kept at −80° C. until ready for processing.

Cells were washed twice in ice-cold PBS supplemented with 5 mM sodium butyrate (Sigma-Aldrich, St. Louis Mo.). Cells were then resuspended in Triton Extraction Buffer (TEB: PBS containing 0.5% TritonX 100(v/v), 2 mM phenylmethylsulfonyl fluoride (PMSF), 0.02% (w/v) sodium azide) also supplemented with 5 mM sodium butyrate at density of 10⁷ cells per mL. Cells were lysed on ice for 10 minutes with gentle agitation. Samples were spun 6,500×g for 10 minutes at 4° C. in order to pellet the nuclei, and supernatants were discarded. The remaining nuclei were then washed with half the volume of TEB and centrifuged again as described previously. The pellet was then resuspended in 0.2N HCl at a density of 4×10⁷ nuclei per mL. Histones were extracted overnight at 4° C. with agitation. Extracts were centrifuged at 6,500×g for 10 minutes at 4° C. and supernatant were saved for analyses or stored at −20° C. Histone extracts were subsequently quantified using a detergent compatible (“DC”) protein assay kit (BioRad, Hercules, Calif.).

For Western Blot analysis, histone extracts were prepared with NuPAGE® 4×LDS Sample Buffer (Life Technologies, Carlsbad, Calif.) and heated to 70° C. for 10 minutes. Samples were run on NuPage® Novex 4-12% Bis-Tris gel (Life Technologies, Carlsbad, Calif.) using MES buffer (Life Technologies, Carlsbad, Calif.). Gels were transferred onto nitrocellulose membranes using the iBlot® system (Life Technologies, Carlsbad, Calif.) on setting P3 for 7 minutes. The antibodies used are shown below in Table 1, along with appropriately labeled secondary detection antibodies.

TABLE 1

| Antigen | Vendor | Catalog Number | Host |
|---|---|---|---|
| H3K9me2 | Abcam (Cambridge, MA) | ab1220 | Mouse |
| Total H3 | Cell Signaling Technology | 9715 | Rabbit |
| | (Beverly, MA) | | |

This experiment shows that IDH1 and IDH2 mutations (IDH1-R132H, IDH1-R132C, and IDH2-R172K) increase the levels of histone methylation of H3K9me2 as compared to WT, as shown in FIG. 1A. FIG. 1B shows that 2-HG is sufficient to increase H3K9me2 levels, thus suggesting that this gain of function would be seen with any IDH1 or IDH2 mutation that produces 2-HG.

### Example 2

### H3K9me2 is Decreased in IDH1 Mutant Cells Upon IDH1 Knockdown or Inhibition Using an IDH1 Mutant-Selective Compound

HT1080 is a fibrosarcoma containing an IDH1 mutant (IDH1-R132C/WT). The HT1080 line was obtained from ATCC (ATCC #CCL-121) and cultured in EMEM supplemented with 10% Tetracycline-Free Fetal Bovine Serum. IDH1 shRNAs were cloned into pLKO-Tet-ON-puromycin vectors (Wiederschain et al., Cell Cycle 2009;8(3):498-504). All shRNAs were validated to give sufficient knockdown of the target at the level of RNA, protein, and 2-HG production. Lentiviral particles were produced by co-transfecting the shRNA plasmid of interest with packaging plasmids Δ8.9 and VSVG into 293T cells (ATCC CRL-11268) using Transit293® transfection reagent (Mirus, Madison, Wis.). Lentiviral supernatants were then used to spin-infect HT-1080 cells in the presence of 8 μg/mL polybrene. Cells recovered overnight in fresh medium and were then selected and continually cultured in the appropriate antibiotics.

The shRNA sequences used in these experiments are listed below in Table 2.

TABLE 2

| shRNA name | Sequence |
|---|---|
| shIDH1-1287 | 5′ GCCTGGCCTGAATATTATACT 3′ |
| | (SEQ ID NO: 5) |
| shIDH1-2073 | 5′ GGAATCCGGAATAAATACTAC 3′ |
| | (SEQ ID NO: 6) |
| shIDH1-2134 | 5′ GCCTGGCCTGAATATTATACT 3′ |
| | (SEQ ID NO: 7) |
| shNTC | 5′ GGATAATGGTGATTGAGATGG 3′ |
| | (SEQ ID NO: 8) |

HT1080 stable lines were cultured with and without doxycycline (100 ng/mL final) for 6 days in the absence of selection. The HT1080 parental line was also treated with DMSO control, an inactive compound, and a mutant selective IDH inhibitor (compound 162) at concentrations of 1, 3, and 10 μM. Compounds were replenished every 3 days. At day 6, cells were lifted using trypsin and processed using a previously described histone extraction protocol (Abcam, Cambridge, Mass.).

Histone extracts were prepared with NuPAGE® 4×LDS Sample Buffer (Life Technologies, Carlsbad, Calif.) and heated to 70° C. for 10 minutes. Samples were run on NuPage® Novex 4-12% Bis-Tris gel (Life Technologies, Carlsbad, Calif.) using MES buffer (Life Technologies, Carlsbad, Calif.). Gel was then transferred onto nitrocellulose membrane using the iBlot® system (Life Technologies, Carlsbad, Calif.) on setting P3 for 7 minutes. The antibodies used are shown above in Table 1.

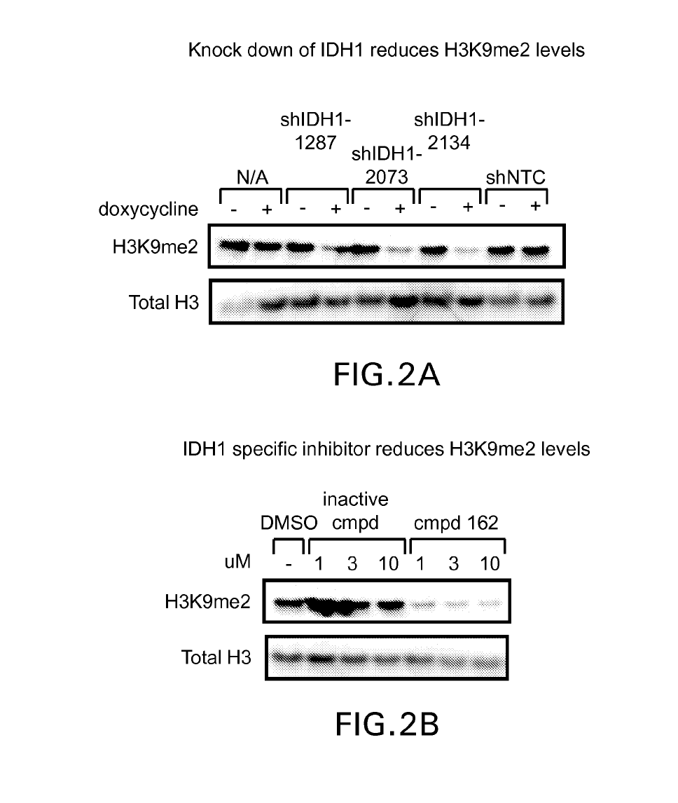

This result is shown in FIG. 2A and indicates that loss of IDH1 function by inducible hairpins is able to reduce H3K9me2 levels in vitro. FIG. 2B shows that a compound specific for IDH1 mutations is also able to reduce methylation of H3K9me2. This data, taken together, further demonstrates a relationship of H3K9 methylation level with mutant IDH1.

### Example 3

### H3K9me2 is Reduced by IDH2 Knockdown in Mutant IDH2 Cancer Cells

SW1353 chondrosarcoma cells (ATCC HTB-94) contain an activating mutation in IDH2 (IDH2-R172S/WT). These cells were cultured in RPM-1640 (Life Technologies, Carlsbad, Calif.) supplemented with 1% sodium pyruvate and 10% Tet-Free fetal bovine serum. IDH2 shRNAs were cloned into pLKO-Tet-ON-puromycin vectors. All shRNAs were validated to give sufficient knockdown of target as judged by RNA, protein, and reduced 2-HG production.

Lentiviral particles were produced by co-transfecting the shRNA plasmid of interest with packaging plasmids Δ8.9 and VSVG into 293T cells using Transit293® transfection reagent (Mirus, Madison, Wis.). Lentiviral supernatants were then used to spin-infect HT-1080 cells in the presence of 8 μg/mL polybrene. Cells recovered overnight in fresh medium and were then selected and continually cultured in appropriate antibiotics.

The shRNA sequences used in these experiments are listed below in Table 3.

TABLE 3

| shRNA | |
|---|---|
| name | Sequence |
| shIDH2-309 | 5′ GTGGACATCCAGCTAAAGTAT 3′ |
| | (SEQ ID NO: 9) |
| shIDH2-891 | 5′ ATCTTTGACAAGCACTATAAG 3′ |
| | (SEQ ID NO: 10) |
| shNTC | 5′ GGATAATGGTGATTGAGATGG 3′ |
| | (SEQ ID NO: 11) |

SW1353 stable lines were cultured with and without doxycycline (100 ng/mL final concentration) for 6 days in the absence of selection. At day 6, cells were lifted using trypsin and histones were extracted as previously described.

Histone extracts were prepared with NuPAGE® 4×LDS Sample Buffer (Life Technologies, Carlsbad, Calif.) and heated to 70° C. for 10 minutes. Samples were run on NuPage® Novex 4-12% Bis-Tris gel (Life Technologies, Carlsbad, Calif.) using MES buffer (Life Technologies, Carlsbad, Calif.). Gel was then transferred onto nitrocellulose membrane using the iBlot® system (Life Technologies) on setting P3 for 7 minutes. The antibodies used are shown above in Table 1.

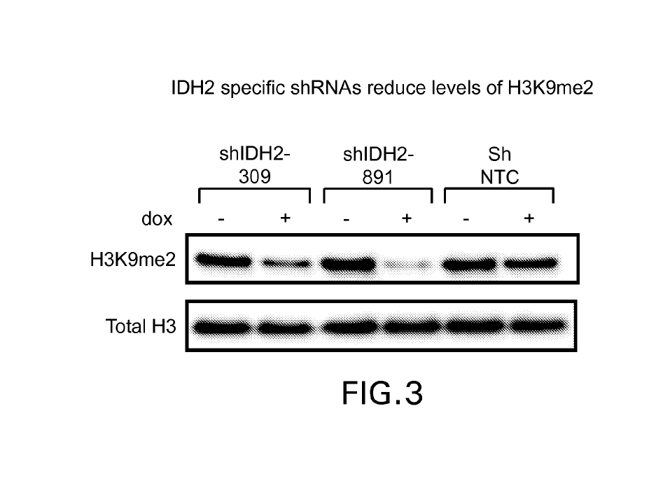

Upon knockdown of IDH2 by shRNA, H3K9me2 methylation level was reduced. This can be seen in FIG. 3, for example, in the shIDH2-891+dox lane. Together with the above data, this demonstrates H3K9me2 levels to be a good pharmacodynamic marker for mutant IDH regardless of cell lineage or whether the mutation occurs in IDH1 or IDH2.

### Example 4

### IDH Inhibitors do not Affect H3K9me2 Levels in an IDH1 and IDH2 Wild Type Cell Line

In order to examine the role of H3K9me2 levels as marker, cells that were wild type for IDH1 and IDH2, for example HCT116 cells, were treated with a compound that inhibits IDH1 mutant activity (compound 162). HCT116 colon cells cancer cells (ATCC CCL-247) were cultured in McCoy's 5A (Life Technologies, Carlsbad, Calif.) supplemented with 10% fetal bovine serum (Hyclone, Logan Utah). These cells were treated for 3-6 days with increasing concentrations of compound 162 or DMSO vehicle as control.

Histone extracts were prepared with NuPAGE® 4×LDS Sample Buffer (Life Technologies, Carlsbad, Calif.) and heated to 70° C. for 10 minutes. Samples were run on NuPage® Novex 4-12% Bis-Tris gel (Life Technologies, Carlsbad, Calif.) using MES buffer (Life Technologies, Carlsbad, Calif.). The gel was then transferred onto nitrocellulose membrane using the iBlot system (Life Technologies, Carlsbad Calif.) on setting P3 for 7 minutes. The antibodies used are shown above in Table 1.

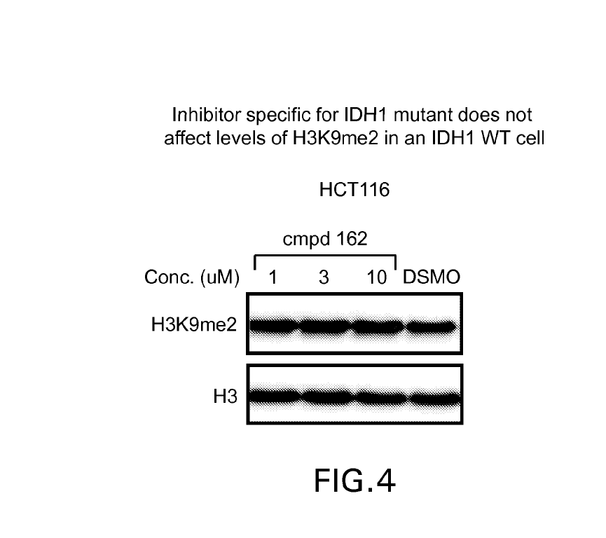

As shown in FIG. 4, compound 162 does not affect H3K9me2 levels in an IDH wild-type cell line, further demonstrating that changes in H3K9me2 level by IDH mutant inhibitor compounds is a selective phenotype observed in IDH mutant cells only.

### Example 5

### Reduction of H3K9me2 by an IDH1 Mutant-Selective Inhibitor is Attenuated by Mutant IDH2 Expression

As disclosed in Example 4, compound 162 is specific for IDH1 mutations. As such, it is a useful compound to determine the contribution of IDH2 to H3K9me2 methylation levels. SNU1079 cholangiocarcinoma cells (KCLB) contain an IDH1 mutation (IDH1-R132C/WT). These cells were cultured in RPMI-1640 (Life Technologies, Carlsbad, Calif.) supplemented with 1% Sodium Pyruvate and 10% Tet-free fetal bovine serum (Hyclone, Logan Utah). Full length IDH2 cDNAs (WT or R172K) were cloned into pLKO-TREX-neomycin for lentiviral transduction (Wee et al., Proc. Nat. Acad. Sci. U.S.A. 2008 105(35):13057-13062). Lentiviral particles were produced by co-transfecting the cDNA expression vector of interest with packaging plasmids Δ8.9 and VSVG into 293T cells (ATCC CRL-11268) using Transit293® transfection reagent (Mirus, Madison, Wis.). Lentiviral supernatants were then used to spin-infect SNU1079 cells in the presence of 8 μg/mL polybrene. Cells recovered overnight in fresh medium and were then selected and continually cultured in appropriate antibiotics.

SNU1079 stably expressing IDH2 isoforms were co-treated with doxycycline (100 ng/mL) final concentration to induce IDH2 (WT or R172K) expression. These lines were treated with DMSO or doses of compound 162. Cells were harvested on day 6 post-treatment and processed for histones as described above.

Histone extracts were prepared with NuPAGE® 4×LDS Sample Buffer (Life Technologies, Carlsbad, Calif.) and heated to 70° C. for 10 minutes. Samples were run on NuPage® Novex 4-12% Bis-Tris gel (Life Technologies, Carlsbad, Calif.) using MES buffer (Life Technologies, Carlsbad, Calif.). The gel was then transferred onto nitrocellulose membrane using the iBlot® system (Life Technologies, Carlsbad, Calif.) on setting P3 for 7 minutes. The antibodies used are shown above in Table 1.

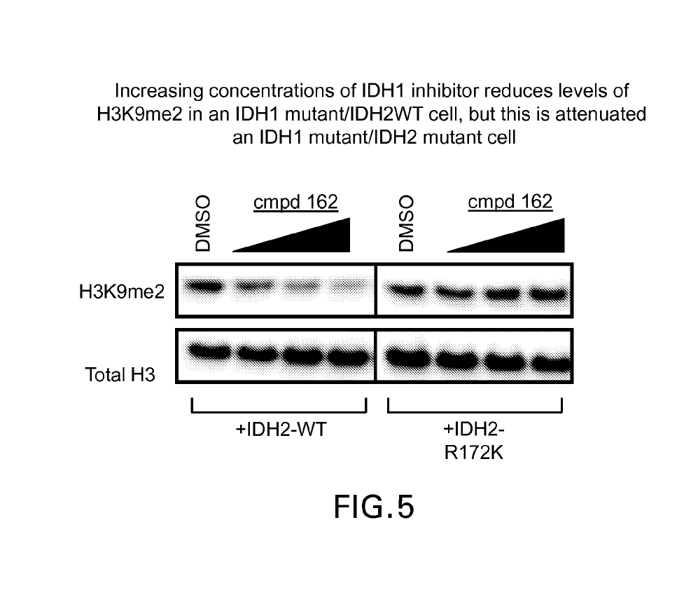

As shown in FIG. 5, compound 162 is able to reduce the H3K9me2 level in a dose-dependent manner in a cell containing an IDH1 mutant(IDH1-R132C/WT) with an IDH2-WT background. Compound 162 is acting on the IDH1 mutant to reduce the levels of H3K9me2, and the IDH2 WT does not affect the levels of H3K9me2, so what is seen is an H3K9me2 level reduction (FIG. 5, left panel). However, the reduction of H3K9me2 level is attenuated by the expression of a gain of function IDH2-R172K mutation as the background (FIG. 5, right panel). As described in Example 1, this IDH2 mutation increases H3K9me2 level. Thus, compound 162 acts on the IDH1 mutation to reduce levels of H3K9me2, but the IDH2 mutation overwhelms this effect. In conclusion, this experiment confirms that sustained production of 2-HG via any mutant IDH isoform is going to increase H3K9me2 level. This also confirms that H3K9me2 levels provide an accurate readout of the pharmacodynamics of IDH inhibitors.

### Examle 6

### Immunohistochemistry Demonstrates a Reduction in H3K9me2 Level

In order to determine if changes in H3K9me2 level could be detected via immunohistochemistry, HT1080 cell lines were plated onto 225 cm³ tissue culture flasks (Corning, Cat #3001, Tewksbury, Mass.). The cells were treated with DMSO or 5 μM compound 162 and incubated for 6 days, with media change at day 3. HT1080 cell pellets were harvested by removing media, washing with 1×PBS, and adding 10 ml of 10% neutral buffer formalin. Cells were then scraped from the bottom of the flask and placed in a 50 ml conical tube, filled to ˜50 ml with 10% neutral buffered formalin, and allowed to fix for 1-2 hours. The conical tube containing the fixed cells was then centrifuged at 1200 rpm for 5 min, and the pelleted cells were wrapped in lens paper, placed in a histology cassette, processed and paraffin embedded.

FFPE slides were cut at 3.5 μm thickness, mounted on charged slides (Thermo-Scientific Colormark® Plus Cat #CM-5951, West Palm Beach, Fla.) and baked at 60° C. for 1 hour. Slides were loaded on the Ventana Discovery XT® Immunostainer. The protocol included a deparaffinization step, using Ventana EZ Prep® (Ventana, Cat #950-100, Tucson, Ariz.). No antigen retrieval was required. The histone H3K9me2 antibody (see Table 1) was diluted in DAKO Cytomation ® Antibody Diluent (DAKO Cat #S0809, Carpenteria, Calif.) at 1:100, manually applied during the primary antibody titration step at a volume of 100 μl, and incubated for 60 minutes at room temperature. The secondary antibody, Ventana OmniMap® HRP-conjugated anti-mouse (Ventana Cat #760-4310 Tucson, Ariz.), was incubated for 4 minutes and detected with Ventana's ChromoMap® DAB detection kit (Ventana Cat #760-159, Tucson, Ariz.). Slides were counterstained for 4 minutes with Ventana Hematoxylin (Ventana Cat #760-2021, Tucson, Ariz.), followed by Ventana Blueing Reagent (Ventana Cat #760-2037, Tucson, Ariz.) for 4 minutes. Slides were then cleared with xylene and cover slipped with Permaslip (Alban Scientific, Cat #A325A, St. Louis, Mo.).

Coverslipped slides were scanned with Aperio ScanScope ® XT (Vista, Calif.) for quantitative digital image analysis. Digital images were analyzed with Visiopharm® (Hoersholm, Denmark) image analysis software. The nuclear detection algorithm detected both hematoxylin and DAB-stained nuclei in the image. Percent of positive nuclei were calculated per the following formula: (% Positive nuclei=# of DAB stained nuclei/# of hematoxylin stained nuclei*100).

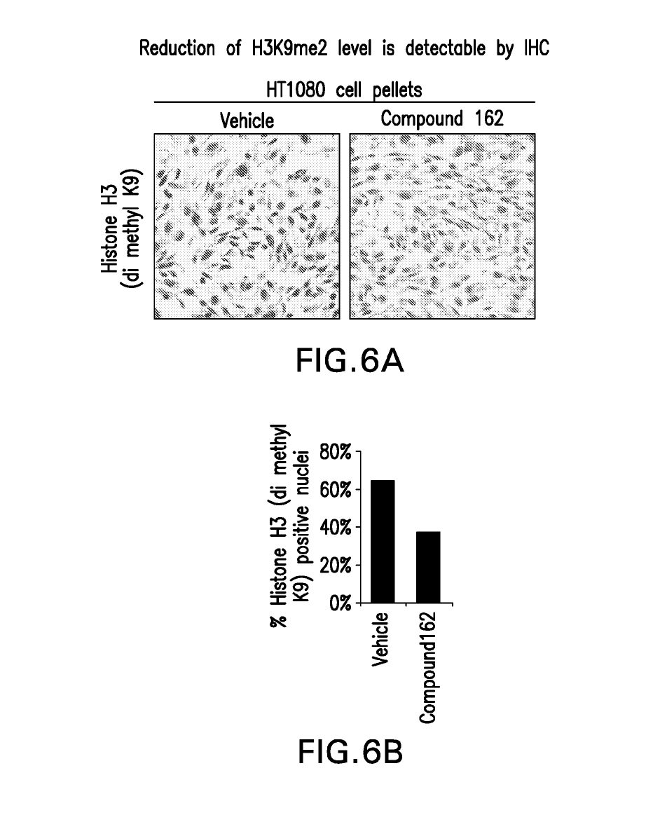

FIG. 6A demonstrates that treatment of IDH1-R132C mutant HT1080 sarcoma cells with compound 162, decreases levels H3K9me2 and the change in level is detectable by immunohistochemistry. When subjected to quantitative image analysis, the entire cross section of the compound 162 treated cells shows a 27% decrease in H3K9me2 levels (FIG. 6B), consistent with what is seen in Western blotting.

### Example 7

### Histone Profiling Demonstrates H3K9me2 is a Robust IDH Mutant Biomarker

Also profiled were three endogenously mutant cell lines upon modulation of the target by chemical or genetic means in order to evaluate other lysine methylation marks. We used histone acid extracts (as described in Example 1) from HT1080 and SNU1079 cells treated with 3 μM compound 162 or DMSO control for 6 days, as well as SW1353 cells that have been induced (100 ng/mL doxycycline) with IDH2 specific shRNA (shIDH2-891 or shNTC) for 6 days. Histones were further enriched with ActiveMotif® Histone purification mini kit (ActiveMotif Inc., Carlsbad, Calif.)

The purified histones were desalted by off line reversed phase chromatography on an 1200 Agilent tower (Agilent, Santa Clara, Calif.) using a Jupiter® 5 μm C4 300 Å Column 150×2 mm (Phenomenex, Torrance, Calif.). The resulting peak area was used to estimate concentration against the peak area of histones of known concentration. The desalted histones were lyophilyzed prior to lysine derivatization.

HeLa cells grown in RPMI 1640 SILAC heavy arginine (¹³C₆¹⁵N₄) media (Life Technologies, Carlsbad, Calif./Cambridge Isotope Laboratories, Andover, Mass.) cell culture aliquots with a cell count of 5×10⁶ cells were treated as above and used as a spiked in process control for sample preparation, derivatization and trypsin digestion.

Free lysines and N-terminus were derivatized using NHS-propionyl synthesized in house, at neutral pH conditions. After the initial derivatization, samples were lyophilized and resuspended for trypsin digestion (Promega, Madison, Wis.). Trypsin digestion completion, due to derivatization, generates proteolytic cuts at C-terminal arginines suitable for LC-MS/MS analysis. Samples were lyophilized and derivatized once more at the peptide level to obtain a homogenous population of derivatized lysines and new N-termini.

Once this process was completed, samples were lyophilized and resuspended for high resolution—high mass accuracy LC-MS/MS in a Thermo Orbitrap Elite® (Thermo Scientific West Palm Beach, Fla.) equipped with an on line nanoAcquity® LC tower (Waters Corp., Milford Mass.) with an HSS T3 1.7 micron column 1×100 mm (Waters Corp. Milford, Mass.).

The data obtained via mass spectrometry was interpreted using Mascot Distiller® (Matrix Science, Boston, Mass.) for identification followed by manual verification. The peak area obtained from the data acquired was processed using Skyline Software® (v1.4.0.422 University of Washington, Seattle, Wash.) for quantitation of the peptides bearing post translational modifications.

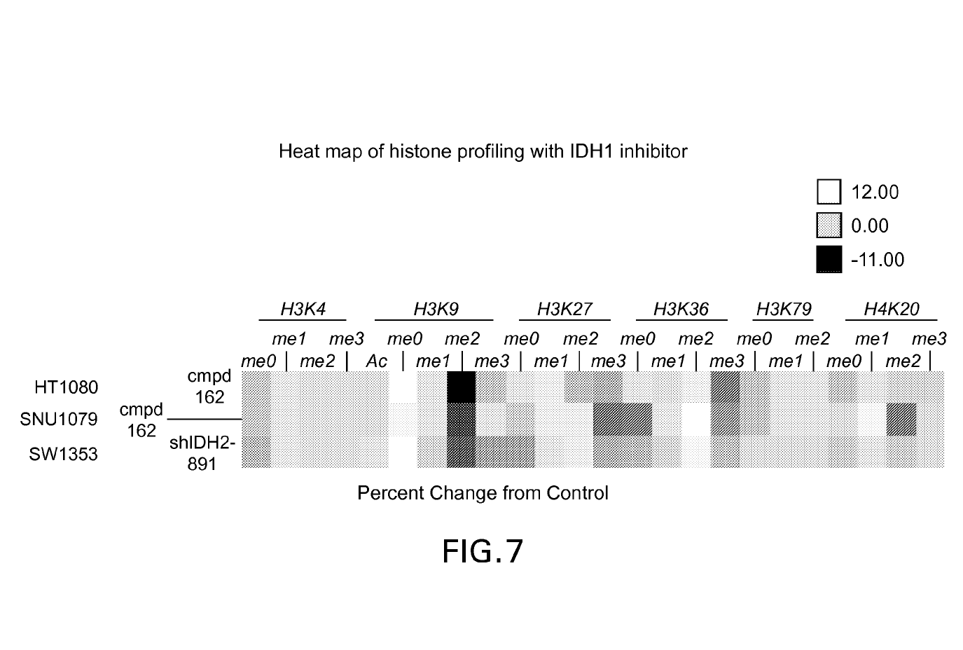

FIG. 7 shows the percent change from control in a heat map format (compound 162 vs. DMSO; IDH2 shRNA vs. non-targeting control shRNA). As can be seen from the heat map representation, the experiment examines five lysines across histone H3 (H3K4, H3K9, H3K27, H3K36 and H3K79) and one lysine on histone H4 (H4K20). These data show that H3K9me2 level reduction and H3K9me0 level increases are the most consistently observed and most robust changes across 3 different cell lineages, IDH mutation type, and IDH inhibitor used (compound vs. shRNA). Furthermore, this shows that the effects observed with H3K9me2 immunodetection are also observed using a non-antibody-based method.

What is claimed is:

1. A method of detecting cancer in a patient, the method comprising:
a) obtaining a cancer sample from a patient; b) sequencing for the presence of a isocitrate dehydrogenase (IDH) mutation in the cancer sample; c) comparing the IDH mutation sequence to an IDH sequence in a non-cancerous or normal patient sample; and d) assaying for the level of di-methylation (me2) of histone H3 at lysine 9 (H3K9me2) in the cancer sample with an IDH mutation and comparing it with the level of H3K9me2 of a non-cancerous or normal patient sample, and a higher level of H3K9me2 in the cancer sample compared to the non-cancerous or normal patient sample is indicative of cancer.

2. The method of claim 1, wherein the IDH mutation is a mutation in IDH1 and is an arginine to histidine change at amino acid position 132 (IDH1-R132H).

3. The method of claim 1, wherein the IDH mutation is a mutation in IDH1 and is an arginine to cysteine change at amino acid position 132 (IDH1-R132C).

4. The method of claim 1, wherein the IDH mutation is a mutation in IDH2 and is an arginine to lysine change at amino acid position 172 (IDH2-R172K)

5. The method of claim 1, wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

6. A method of assaying for the response of a patient to treatment with an IDH inhibitor, the method comprising: a) obtaining a cancer sample from a patient prior to administration of an IDH inhibitor; b) administration to a patient of at least one IDH inhibitor; c) assaying for a level of H3K9me2 in the sample obtained from the patient who has been administered the IDH inhibitor; and d) comparing the level of H3K9me2 in the cancer sample taken prior to administration of the IDH inhibitor or the level of H3K9me2 in a non-cancerous or control sample.

7. The method of claim 6, wherein the level of H3K9me2 is reduced.

8. The method of claim 6, wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

9. The method of claim 6, wherein the IDH inhibitor inhibits IDH1.

10. The method of claim 6, wherein the IDH inhibitor inhibits an IDH1 mutant, and the IDH1 mutation is an arginine to histidine change at amino acid position 132 (IDH1-R132H).

11. The method of claim 6, wherein the IDH inhibitor inhibits an IDH1 mutant, and the IDH1 mutation is an arginine to cysteine change at amino acid position 132 (IDH1-R132C).

12. The method of claim 6, wherein the IDH inhibitor is an oxazolidinone.

13. The method of claim 6, wherein the IDH inhibitor inhibits an IDH2 mutant, and the IDH2 mutation is an arginine to lysine change (IDH2-R172K).

14. The method of claim 6, wherein the IDH inhibitor is administered at different time points.

15. The method of claim 6, wherein assaying for the level of H3K9me2 in the cancer sample is measured at least at two different time points.

16. The method of claim 6, wherein the steps c) and d) are repeated at 1 hour, 2 hours, 3, hours, 4, hours, 8 hours, 16 hours and 48 hours.

17. The method of claim 6, wherein assaying for the level of H3K9me2 is done by mass spectrometry.

18. The method of claim 6, wherein assaying for the level of H3K9me2 is done by Western blotting.

19. A method of screening for an IDH inhibitor candidate, the method comprising: a) contacting a cell containing an IDH mutation with an IDH inhibitor candidate; b) assaying for a level of H3K9me2; and c) comparing the level of H3K9me2 from the IDH mutant cell contacted with the IDH inhibitor candidate with the level of H3K9me2 of a normal or control cell and/or untreated cell containing the IDH mutation.

20. The method of claim 19, wherein the IDH inhibitor inhibits IDH1.

21. The method of claim 19, wherein the IDH inhibitor inhibits an IDH1 mutant, and the IDH1 mutation is an arginine to histidine change at amino acid position 132 (IDH1-R132H).

22. The method of claim 19, wherein the IDH inhibitor inhibits an IDH1 mutant, and the IDH1 mutation is an arginine to cysteine change at amino acid position 132 (IDH1-R132C).

23. The method of claim 19, wherein the IDH inhibitor inhibits an IDH2 mutant, and the IDH2 mutation is an arginine to lysine change (IDH2-R172K).

24. The method of claim 19, wherein the cell containing an IDH mutation is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

25. The method of claim 19, wherein assaying for the level of H3K9me2 is done by mass spectrometry.

26. The method of claim 19, wherein assaying for the level of H3K9me2 is done by Western blotting.

27. A composition comprising H3K9me2 for use in diagnosing a patient response in a selected cancer patient population, wherein the cancer patient population is selected on the basis of (i) having increased levels of H3K9me2 in a cancer cell sample obtained from said patients compared to a normal control cell sample, and (ii) H3K9me2 levels are reduced upon administration of an IDH inhibitor.

28. The composition of claim 27, wherein the IDH inhibitor inhibits IDH1.

29. The composition of claim 27, wherein the IDH inhibitor inhibits an IDH1 mutant, wherein the mutation in IDH1 is an arginine to histidine change at amino acid position 132 (IDH1-R132H).

30. The composition of claim 27, wherein the IDH inhibitor inhibits an IDH1 mutant, wherein the mutation in IDH1 is an arginine to cysteine change at amino acid position 132 (IDH1-R132C).

31. The composition of claim 27, wherein the IDH inhibitor inhibits an IDH2 mutant, wherein the mutation in IDH2 is an arginine to lysine change (IDH2-R172K).

32. The composition wherein the cancer sample is selected from the group consisting of: low grade glioma, glioblastoma multiforme, acute myeloid leukemia, myelodysplastic syndrome, peripheral T-cell lymphoma, cholangiocarcinoma, chondrosarcoma, cartilaginous cancer associated with Ollier Disease, cartilaginous cancer associated with Mafucci Syndrome, prostate cancer, lung cancer, colon cancer, melanoma, supratentorial primordial neuroectodermal tumors and breast cancer.

33. A kit for predicting the response of a cancer patient to treatment with an IDH inhibitor comprising: i) means for detecting H3K9me2; and ii) instructions how to use said kit.
